(19) United States
(12) Patent Application Publication
(10) Pub. No.: US 2015/0085092 A1
(43) Pub. Date: Mar. 26, 2015

(54) SURGERY ASSISTANCE DEVICE AND SURGERY ASSISTANCE PROGRAM

(71) Applicants: Panasonic Healthcare Co., Ltd., Ehime (JP); Panasonic Medical Solutions Co., Ltd., Osaka (JP)

(72) Inventors: Tomoaki Takemura, Osaka (JP); Ryoichi Imanaka, Osaka (JP); Keiho Imanishi, Hyogo (JP); Munehito Yoshida, Wakayama (JP); Masahiko Kioka, Osaka (JP)

(73) Assignees: Panasonic Healthcare Co., Ltd., Ehime (JP); Panasonic Medical Solutions Co., Ltd., Osaka (JP)

(21) Appl. No.: 14/387,146

(22) PCT Filed: Mar. 26, 2013
(86) PCT No.: PCT/JP2013/002062

§ 371 Date: Sep. 22, 2014

(30) Foreign Application Priority Data
Mar. 29, 2012 (JP) 2012-077118

(51) Int. Cl. A61B 19/00; H04N 5/232

(52) U.S. Cl. CPC A61B 19/50; H04N 5/232; A61B 19/56; A61B 2019/562; A61B 2019/501
USPC 348/65

(57) ABSTRACT

A personal computer (1) comprises a tomographic image information acquisition section (6), a memory (9), and a volume rendering computer (13). The tomographic image information acquisition section (6) acquires tomographic image information. The memory (9) is connected to the tomographic image information acquisition section (6) and stores voxel information related to tomographic image information. The volume rendering computer (13) is connected to the memory (9), samples voxel information in a direction perpendicular to the sight line on the basis of voxel information, and sets a restricted display area and an endoscopic display area acquired by the endoscope and produced by the volume rendering computer (13), and displays these on a display (2).

### TECHNICAL FIELD

The present invention relates to a surgery assistance device and a surgery assistance program that are used when a health care provider performs a simulation of surgery.

### BACKGROUND ART

In a medical facility, surgery assistance devices that allow surgery to be simulated are employed in order to perform better surgery.

A conventional surgery assistance device comprised, for example, a tomographic image information acquisition section for acquiring tomographic image information, such as an image acquired by PET (positron emission tomography), a nuclear magnetic resonance image (MRI), or an X-ray CT image, a memory connected to the tomographic image information acquisition section, a volume rendering computer connected to the memory, a display for displaying the computation results of the volume rendering computer, and an input section for giving resecting instructions with respect to a displayed object that is being displayed on the display.

For example, Patent Literature 1 discloses an endoscopic surgery assistance device with which a tomographic image acquired by an MRI device, a CT device, or another such imaging device is used to assist endoscopic surgery by providing a display image.

### CITATION LIST

### Patent Literature

Patent Literature 1: Japanese Patent No. 4,152,402

### SUMMARY

However, the following problems were encountered with the above-mentioned conventional surgery assistance device.

Specifically, with the surgery assistance device disclosed in the above-mentioned publication, it is possible to perform surgery while checking the positional relation between the endoscope and the surgical site by giving the surgeon the position of the surgical site on a displayed image.

However, since these display images are not linked to the surgical plan, it is difficult to ascertain the correct surgical site in a simulation prior to surgery.

The above-mentioned surgical method that makes use of an endoscope generally results in a smaller wound than in a laparotomy or the like, and greatly lessens the burden on the patient. Therefore, endoscopic surgery has in recent years come to be used in many different operations, such as surgery for lumbar spinal stenosis.

In surgery using an endoscope, a tubular member called a retractor is placed in the body of the patient, the endoscope is inserted along this tubular member, and the surgeon performs the surgery while looking at the area around the surgical site on a monitor screen. Thus, what the physician, etc., can see during actual surgery is restricted to a narrower range than in ordinary open surgery, so in the resection simulation carried out prior to surgery, it is preferable for the display to be as close as possible to the state that will be displayed on the monitor screen during actual surgery.

It is an object of the present invention to provide a surgery assistance device and surgery assistance program with which a resection simulation can be carried out while giving a display that approximates the state that will actually be displayed on the display screen, even in surgery involving an endoscope.

The surgery assistance device pertaining to the first invention is a surgery assistance device that displays a simulation image during surgery performed by inserting an endoscope into the interior of a surgical instrument, comprising a tomographic image information acquisition section, a memory, a volume rendering computer, and a display controller. The tomographic image information acquisition section acquires tomographic image information. The memory is connected to the tomographic image information acquisition section and stores voxel information related to the tomographic image information. The volume rendering computer is connected to the memory and samples voxel information in a direction perpendicular to the sight line on the basis of the voxel information. The display controller sets a first display area acquired by the endoscope and produced by the volume rendering computer, and a second display area in which display is restricted by the surgical instrument during actual surgery, and displays the first and the second display areas on a display section.

In a simulation of surgery in which an endoscope is used in a state in which a three-dimensional image produced using a plurality of X-ray CT images, for example, is used to display the area around a certain bone, blood vessel, organ, or the like, the display shows everything up to the portion of the field of view restricted by the surgical instrument into which the endoscope is inserted.

The above-mentioned tomographic image includes, for example, two-dimensional images acquired using a medical device such as X-ray CT, MRI, or PET. The above-mentioned surgical instrument includes tubular retractors into which an endoscope is inserted.

Consequently, when an endoscopic surgery for lumbar spinal stenosis is simulated, for example, the display is masked so that you cannot see the portion restricted by the retractor or other tubular surgical instrument, which allows the simulation to show a state that approximates an actual endoscopic image.

As a result, the display approximates the endoscopic image displayed during actual surgery using an endoscope, so the surgery can be simulated more effectively.

The surgery assistance device pertaining to the second invention is the surgery assistance device pertaining to the first invention, further comprising a display section having first and second display areas.

Here, a monitor or other such display section is provided as a surgery assistance device.

This allows the surgery to be assisted while a simulation image of the above-mentioned endoscopic surgery is displayed on a display section.

The surgery assistance device pertaining to the third invention is the surgery assistance device pertaining to the second invention, wherein the display controller detects and displays, as an insertion limit position, the position on the simulation image where the surgical instrument comes into contact with the boundary of the surgical site in a state in which the surgical instrument has been inserted into the body.

Here, the depth position of the retractor or other such surgical instrument into which the endoscope is inserted, with respect to the surgical site, is sensed, and the position where the surgical instrument comes into contact with a bone or the like around the surgical site is sensed and displayed as the insertion limit position.

Here, in actual endoscopic surgery, the surgery is performed in a state in which the surgical instrument has been inserted up to the position where it touches a bone or the like. Unless the position of the surgical instrument in the depth direction is taken into account, in actual practice display will be possible up to a position where the surgical instrument cannot fit, so this is undesirable in terms of carrying out an accurate surgery simulation.

This prevents an endoscopic image that cannot be seen in actual endoscopic surgery from being displayed by sensing and displaying the insertion limit position in order to sense the positional relation between the surgical instrument and the surgical site and to limit the position of the surgical instrument in the depth direction, which allows the surgical simulation to better approximate an actual endoscopic surgery.

The surgery assistance device pertaining to the fourth invention is the surgery assistance device pertaining to any of the first to third inventions, wherein the endoscope is an oblique-viewing endoscope.

Here, an oblique-viewing endoscope is used as the endoscope used in the endoscopic surgery to be simulated.

Consequently, a simulation of an endoscopic surgery using an endoscope with a wider field of view than a direct-view endoscope can be performed while the user looks at an endoscopic image that approximates the display image during actual surgery.

The surgery assistance program pertaining to the fifth invention is a surgery assistance program for displaying a simulation image during surgery performed by inserting an endoscope into the interior of a surgical instrument, wherein the surgery assistance program comprises an acquisition step, a volume rendering step, and a display step. In the acquisition step, tomographic image information is acquired. In the volume rendering step, voxel information is sampled in a direction perpendicular to the sight line on the basis of voxel information related to the tomographic image information. In the display step, a first display area acquired by the endoscope and produced in the volume rendering step is set, and a second display area in which display is restricted by the surgical instrument during actual surgery is set, and the first and the second display areas are displayed on a display section.

Here, in the simulation of surgery using an endoscope in a state in which a plurality of X-ray CT images are used to display the area around a certain bone, blood vessel, organ, or the like, the display shows everything up to the portion of the field of view restricted by the surgical instrument into which the endoscope is inserted.

The above-mentioned tomographic image includes, for example, two-dimensional images acquired using a medical device such as X-ray CT, MRI, or PET. The above-mentioned surgical instrument includes tubular retractors into which an endoscope is inserted.

Consequently, when an endoscopic surgery for lumbar spinal stenosis is simulated, for example, the display is masked so that you cannot see the portion restricted by the retractor or other tubular surgical instrument, which allows the simulation to show a state that approximates an actual endoscopic image.

As a result, the display approximates the endoscopic image displayed during actual surgery using an endoscope, so the surgery can be simulated more effectively.

### BRIEF DESCRIPTION OF DRAWINGS

FIG. 1 is an oblique view of a personal computer (surgery assistance device) pertaining to an embodiment of the present invention;

FIG. 2 is a control block diagram of the personal computer in FIG. 1;

FIG. 3 is a block diagram of the configuration of an endoscope parameter storage section in a memory included in the control blocks in FIG. 2;

FIG. 4 is a block diagram of the configuration of a surgical instrument parameter storage section in the memory included in the control blocks in FIG. 2;

FIG. 5A is an operational flowchart of the personal computer in FIG. 1, and FIG. 5B is an operational flowchart of the flow in S6 of FIG. 5A;

FIG. 6 is a diagram illustrating a method for automatically detecting the insertion position of a surgical instrument when a tubular surgical instrument (retractor) is used;

FIGS. 7A and 7B are diagrams illustrating mapping from two-dimensional input with a mouse to three-dimensional operation with an endoscope when a tubular surgical instrument (retractor) is used;

FIG. 8 is a diagram illustrating mapping from two-dimensional input with a mouse to three-dimensional operation with an endoscope;

FIG. 9 is a diagram illustrating the display of a volume rendering image that shows the oblique angle of an oblique endoscope;

FIGS. 10A to 10C show the display when the distal end position and sight line vector of an oblique endoscope are shown in a three-panel view;

FIG. 11 shows an oblique endoscopic image displayed by the personal computer in FIG. 1;

FIG. 12A shows an oblique endoscopic image pertaining to this embodiment, and FIG. 12B shows an endoscopic image when a direct-view endoscope is used in place of an oblique endoscope; and

FIG. 13 shows a monitor screen that shows the restricted display area of an endoscopic image.

### DESCRIPTION OF EMBODIMENTS

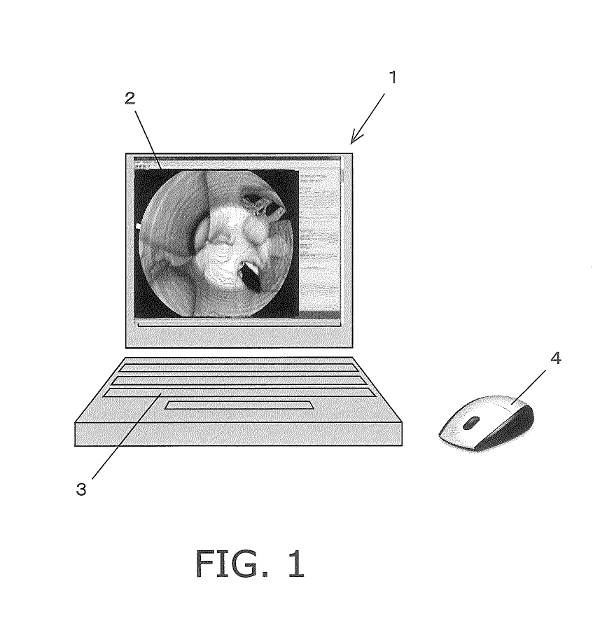

The personal computer (surgery assistance device) pertaining to an embodiment of the present invention will now be described through reference to FIGS. 1 to 13.

In this embodiment, we will describe the simulation of surgery on lumbar spinal stenosis by using an oblique endoscope, but the present invention is not limited to or by this.

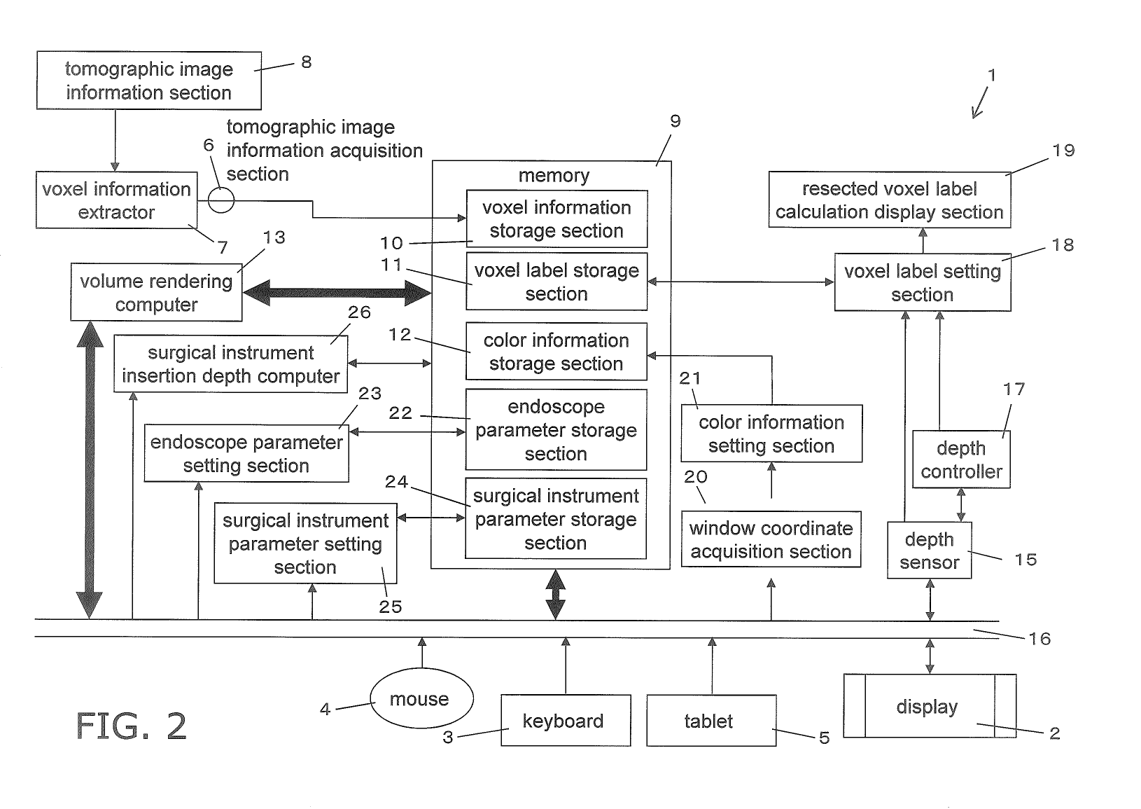

As shown in FIG. 1, the personal computer 1 in this embodiment comprises a display (display component) 2, various input components (a keyboard 3, a mouse 4, and a tablet 5 (see FIG. 2)).

The display 2 displays three-dimensional images of organs or the like formed from a plurality of tomographic images such as X-ray CT images (an endoscopic image is displayed in the example in FIG. 1), and also displays the results of resection simulation.

As shown in FIG. 2, the personal computer 1 has internally formed control blocks, such as a tomographic image information acquisition section 6.

The tomographic image information acquisition section 6 is connected via a voxel information extractor 7 to a tomographic image information section 8. That is, with the tomographic image information section 8, tomographic image information is supplied from a device that captures tomographic images, such as CT, MRI, or PET, and this tomographic image information is extracted as voxel information by the voxel information extractor 7.

A memory 9 is provided inside the personal computer 1, and has a voxel information storage section 10, a voxel label storage section 11, a color information storage section 12, an endoscope parameter storage section 22, and a surgical instrument parameter storage section 24. The memory 9 is also connected to a volume rendering computer 13.

The voxel information storage section 10 stores voxel information received from the voxel information extractor 7 via the tomographic image information acquisition section 6.

The voxel label storage section 11 has a first voxel label storage section, a second voxel label storage section, and a third voxel label storage section. These first to third voxel label storage sections are each provided corresponding to a preset range of CT values (discussed below), that is, to the organ to be displayed. For example, the first voxel label storage section corresponds to a range of CT values for displaying a liver, the second voxel label storage section corresponds to a range of CT values for displaying a blood vessel, and the third voxel label storage section corresponds to a range of CT values for displaying a bone.

The color information storage section 12 has a plurality of internal storage sections. The storage sections are each provided corresponding to a preset range of CT values, that is, a bone, blood vessel, nerve, organ, or the like that is to be displayed. Examples include a storage section corresponding to a range of CT values displaying an organ, a storage section corresponding to a range of CT values displaying a blood vessel, and a storage section corresponding to a range of CT values displaying a bone. Color information that is different for the bone, blood vessel, nerve, or organ to be displayed is provided to each storage section. For example, the range of CT values corresponding to a bone stores white color information, while the range of CT values corresponding to a blood vessel stores red color information.

The CT values set for the bone, blood vessel, or organ to be displayed are numerical representations of the extent of X-ray absorption in the body, and are expressed as relative values (in units of HU), with water at zero. For instance, the range of CT values in which a bone is displayed is 500 to 1000 HU, the range of CT values in which blood is displayed is 30 to 50 HU, the range of CT values in which a liver is displayed is 60 to 70 HU, and the range of CT values in which a kidney is displayed is 30 to 40 HU.

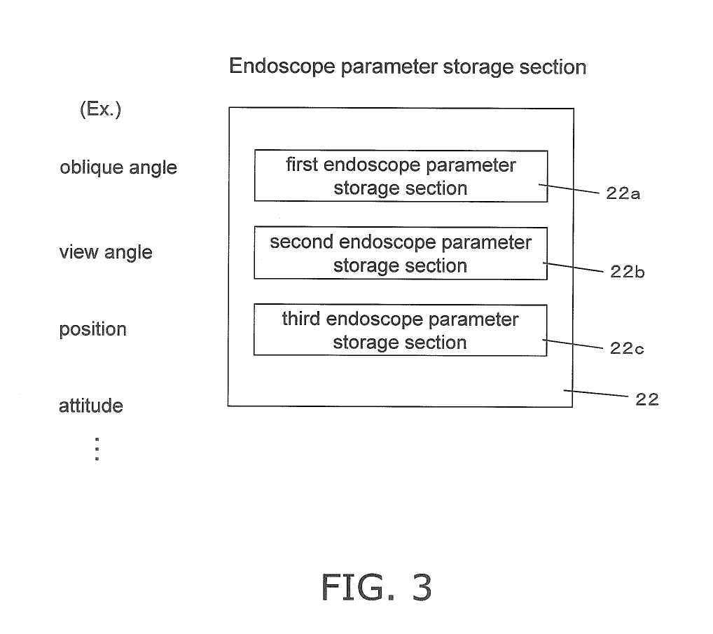

As shown in FIG. 3, the endoscope parameter storage section 22 has a first endoscope parameter storage section 22a, a second endoscope parameter storage section 22b, and a third endoscope parameter storage section 22c. The first to third endoscope parameter storage sections 22a to 22c store endoscope oblique angles, viewing angles, positions, attitudes, and other such information. The endoscope parameter storage section 22 is connected to an endoscope parameter setting section 23, as shown in FIG. 2.

The endoscope parameter setting section 23 sets the endoscope parameters inputted via the keyboard 3 or the mouse 4, and sends them to the endoscope parameter storage section 22.

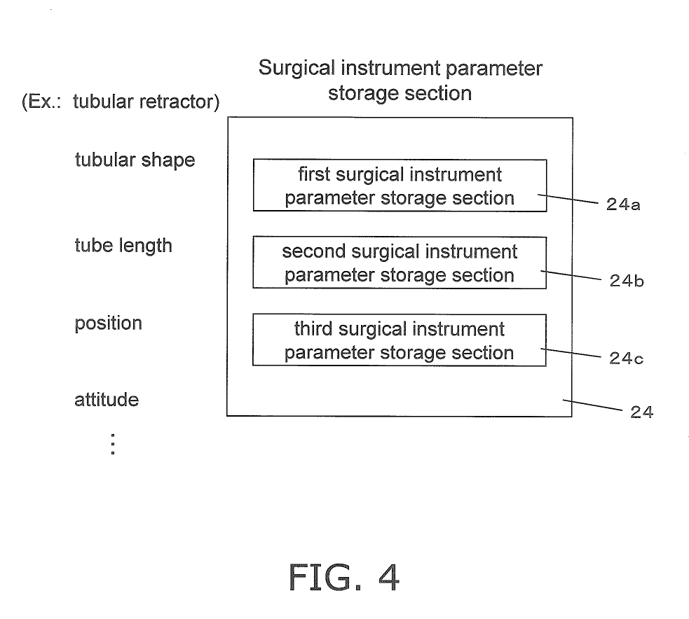

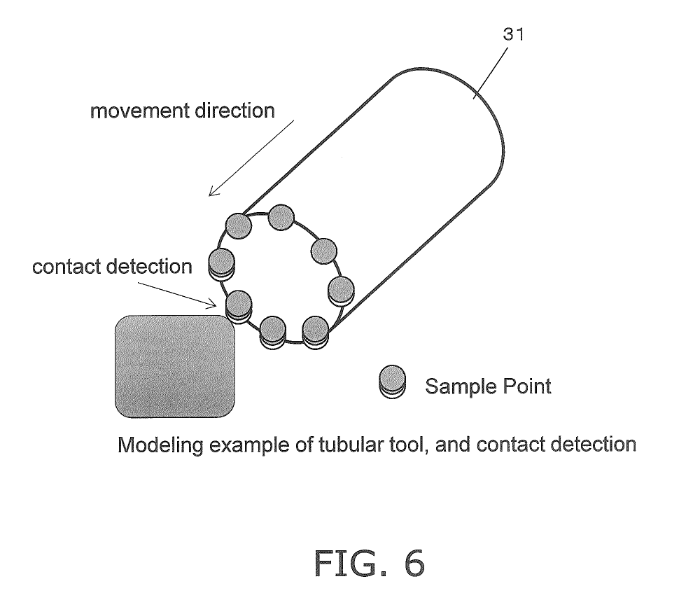

As shown in FIG. 4, the surgical instrument parameter storage section 24 has a first surgical instrument parameter storage section 24a, a second surgical instrument parameter storage section 24b, and a third surgical instrument parameter storage section 24c. The first to third surgical instrument parameter storage sections 24a to 24c each store information such as the shape, length, position, and attitude of the tubular retractor (if the surgical instrument is a tubular retractor (see FIG. 6)), for example. As shown in FIG. 2, the surgical instrument parameter storage section 24 is connected to a surgical instrument parameter setting section 25.

The surgical instrument parameter setting section 25 sets surgical instrument parameters for the retractor, etc., that are inputted via the keyboard 3 or the mouse 4, and sends them to the surgical instrument parameter storage section 24.

A surgical instrument insertion depth computer 26 is connected to the surgical instrument parameter storage section 24 inside the memory 9, and computes the insertion depth of the retractor or other surgical instrument (the depth position at the surgical site).

The volume rendering computer 13 acquires a plurality of sets of slice information at a specific spacing in the Z direction and perpendicular to the sight line, on the basis of the voxel information stored in the voxel information storage section 10, the voxel labels stored in the voxel label storage section 11, and the color information stored in the color information storage section 12. The volume rendering computer 13 then displays this computation result as a three-dimensional image on the display 2.

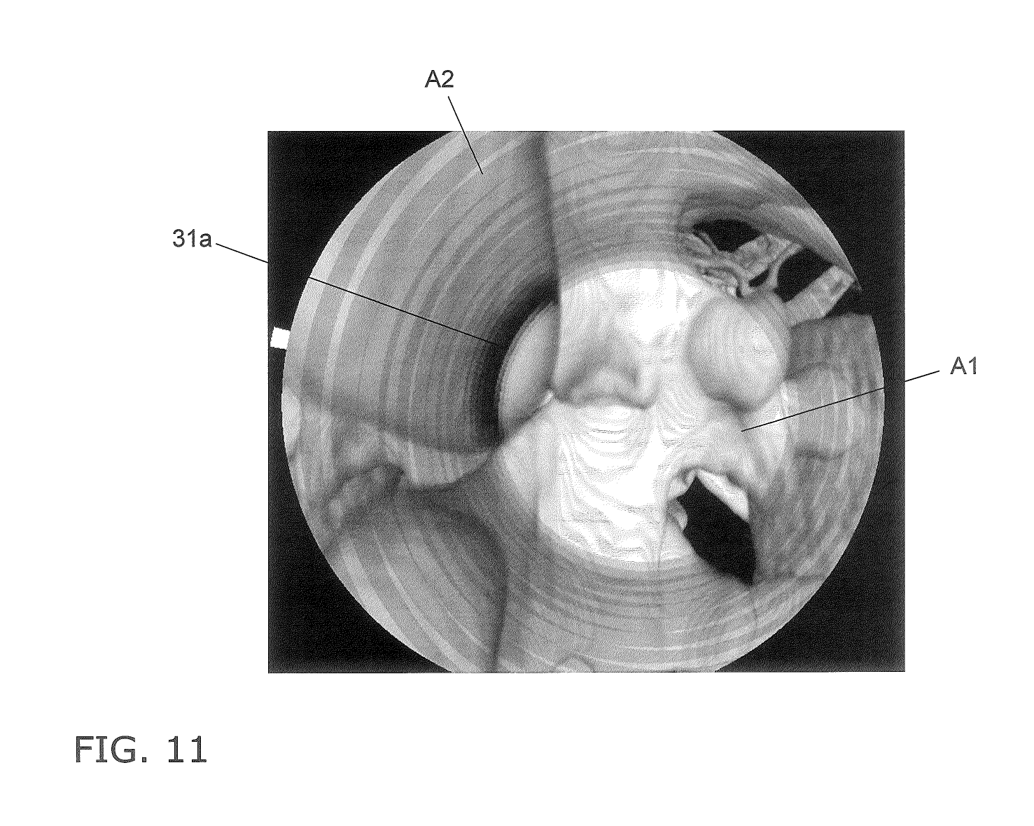

The volume rendering computer 13 also displays an endoscopic image on the display 2 in a masked state that reflects image information in which the field of view is restricted by a retractor or other surgical instrument, with respect to the image information obtained by the endoscope, on the basis of endoscopic information stored in the endoscope parameter storage section 22 and surgical instrument information stored in the surgical instrument parameter storage section 24. More specifically, the volume rendering computer 13 sets an endoscopic image display area (first display area) A1 (see FIG. 11) acquired by the endoscope, and a restricted display area (second display area) A2 (see FIG. 11).

The endoscopic image display area A1 here is a display area that is displayed on the monitor screen of the display 2 during actual endoscopic surgery. The restricted display area A2 is a display area in which the display acquired by the endoscope is restricted by the inner wall portion, etc., of the surgical instrument, such as a tubular retractor, and refers to a region whose display is masked in endoscopic surgery simulation (see FIG. 11).

The volume rendering computer 13 is also connected to a depth sensor 15 via a bus 16.

The depth sensor 15 measures the ray casting scanning distance, and is connected to a depth controller 17 and a voxel label setting section 18.

The voxel label setting section 18 is connected to the voxel label storage section 11 and to a resected voxel label calculation display section 19.

In addition to the above-mentioned volume rendering computer 13 and depth sensor 15, the bus 16 is also connected to a window coordinate acquisition section 20, such as the color information storage section 12 in the memory 9, and displays three-dimensional images and so forth on the display 2 on the basis of what is inputted from the keyboard 3, the mouse 4, the tablet 5, and so on.

The window coordinate acquisition section 20 is connected to the depth sensor 15 and a color information setting section 21.

The color information setting section 21 is connected to the color information storage section 12 in the memory 9.

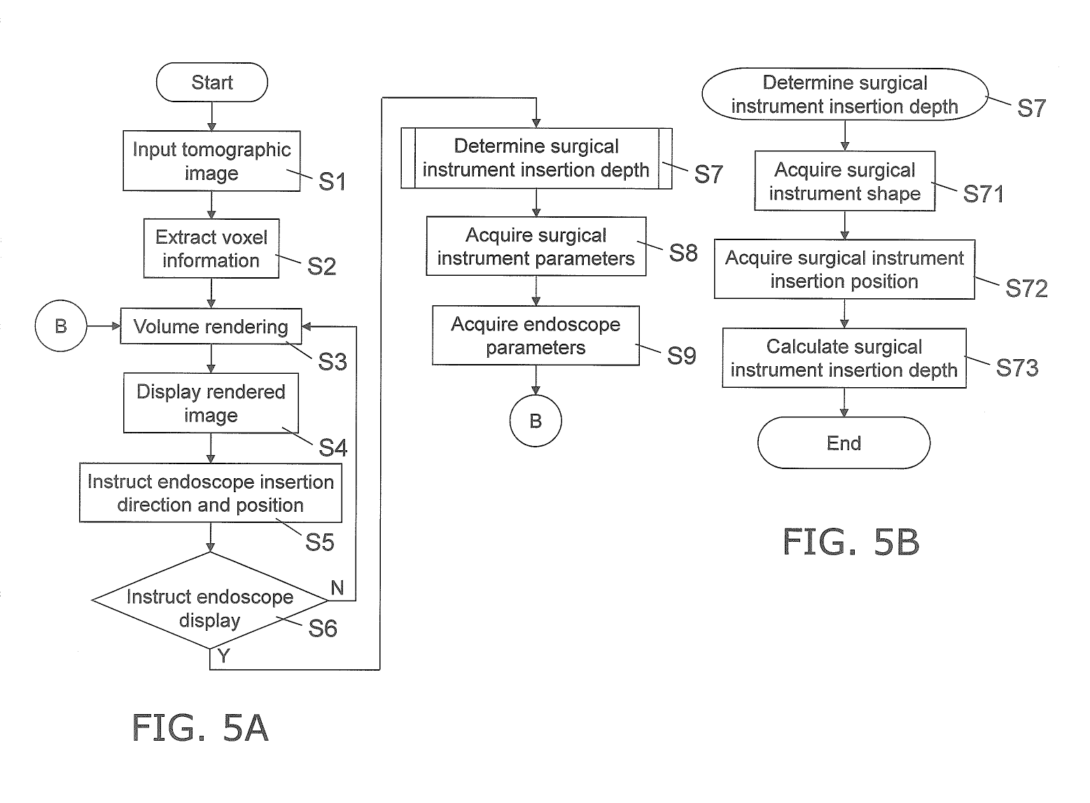

FIGS. 5A and 5B show the control flow, illustrating the operation of the personal computer (surgery assistance device) 1 in this embodiment.

As shown in FIG. 5A, with the personal computer 1 in this embodiment, first in S1, as discussed above, tomographic image information is inputted from the tomographic image information section 8 and supplied to the voxel information extractor 7.

Then, in S2, voxel information is extracted from the tomographic image information by the voxel information extractor 7. The extracted voxel information goes through the tomographic image information acquisition section 6 and is stored in the voxel information storage section 10 of the memory 9. The voxel information stored in the voxel information storage section 10 is information about the points made up of I(x,y,z,α), for example. I here is brightness information about these points, while x, y, and z are coordinate points, and a is transparency information.

Then, in S3, the volume rendering computer 13 calculates a plurality of sets of slice information at a specific spacing in the Z direction and perpendicular to the sight line, on the basis of the voxel information stored in the voxel information storage section 10, and acquires a slice information group. This slice information group is at least temporarily stored in the volume rendering computer 13.

The above-mentioned slice information perpendicular to the sight line refers to a plane that is perpendicular to the sight line. For example, in a state in which the display 2 has been erected vertically, when it is viewed in a state in which it and the plane of the user's face are parallel, the slice information is in a plane perpendicular to the sight line.

The plurality of sets of slice information thus obtained include information about the points made up of I(x,y,z,α), as mentioned above. Thus, the slice information is such that a plurality of voxel labels 14 are disposed in the Z direction, for example. The group of voxel labels 14 is stored in the voxel label storage section 11.

Then, in S4, a rendered image is displayed on the display 2. At this point, the mouse 4 or the like is used to designate the range of CT values on the display 2, and the bone, blood vessel, or the like to be resected is selected and displayed.

Then, in S5, a user instruction regarding the endoscope insertion direction and position is inputted.

Then, in S6, it is determined whether or not an instruction to give an endoscope display has been received from the user. If an endoscope display instruction has been received, the flow proceeds to S7. On the other hand, if an endoscope display instruction has not been received, the flow returns to S3.

Then in S7, the insertion depth of the surgical instrument is determined on the basis of information inputted with the keyboard 3 or the mouse 4.

More precisely, as shown in FIG. 5B, in S71 the surgical instrument insertion depth computer 26 acquires information related to the surgical instrument shape from the surgical instrument parameter storage section 24.

Then, in S72, the surgical instrument insertion depth computer 26 acquires information related to the insertion position of the surgical instrument with respect to the three-dimensional image produced by the volume rendering computer 13 (such as the inside diameter of the retractor, and the distance from the center of the endoscope inside the retractor).

Then, in S73, the surgical instrument insertion depth computer 26 senses the depth position (surgical instrument insertion depth) where there is contact with the bone or other side included in the three-dimensional image after the retractor or other surgical instrument has been inserted, on the basis of the information acquired in S72 (in other words, the insertion limit position is sensed).

Consequently, the limit position to which the retractor or other surgical instrument can be inserted in actual endoscopic surgery is accurately ascertained, which prevents the surgical simulation from being carried out in a state in which the surgical instrument has been inserted to a deeper position than the real insertion limit position. Then, in S8, the volume rendering computer 13 acquires the necessary parameters related to the tubular retractor or other surgical instrument from the surgical instrument parameter storage section 24.

Then, in S9, the volume rendering computer 13 acquires the necessary parameters related to the endoscope from the endoscope parameter storage section 22, and the flow proceeds to S3.

In S3 here, the volume rendering computer 13 sets the endoscopic image display area A1 acquired by the endoscope (see FIG. 11) and the restricted display area A2 (see FIG. 11) out of the three-dimensional image produced by the volume rendering computer 13, on the basis of the surgical instrument parameters and endoscope parameters acquired in S8 and S9, and displays these on the display screen of the display 2.

Specifically, with the personal computer 1 in this embodiment, rather than simply displaying the three-dimensional image produced by the volume rendering computer 13, just the image that can be acquired by the endoscope in actual endoscopic surgery is displayed, and the restricted display area A2 where the display is restricted by the retractor 31 or other surgical instrument is not displayed (see FIG. 11).

Consequently, when endoscopic surgery is simulated, the simulation will show a display mode that approximates the state that is displayed in actual endoscopic surgery. As a result, surgery can be assisted more effectively.

The method for determining the insertion depth of the retractor 31 will now be described through reference to FIG. 5B, and the retractor insertion position automatic sensing function will be described through reference to FIG. 6.

Here, modeling is performed in which a plurality of sampling points are disposed outside of the surgical instrument and the site where contact is expected to occur, on the basis of the retractor diameter, length, movement direction (insertion direction), and other such parameters. More precisely, points of contact with the bone, etc., included in the three-dimensional image in the movement direction are sensed for all the points set at the distal end of the retractor 31, with respect to the three-dimensional image produced by the volume rendering computer 13. Then, the point where the distal end of the retractor 31 makes initial contact with the bone, etc., included in the three-dimensional image is set as the insertion limit position of the retractor 31.

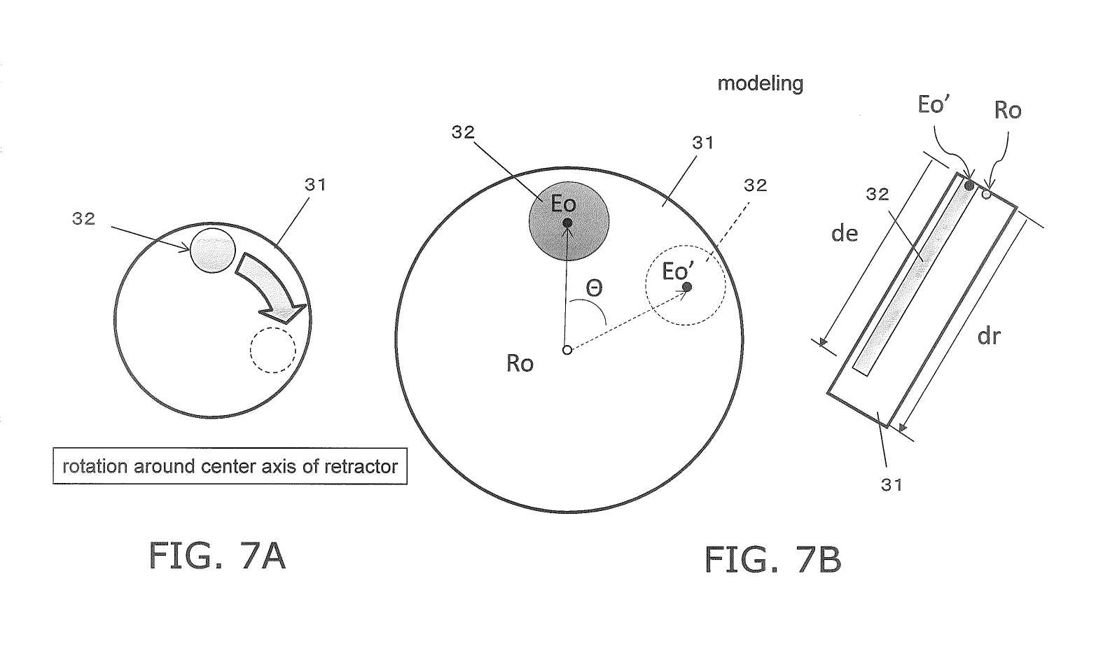

Next, mapping from two-dimensional input with the mouse 4 to three-dimensional input with an endoscope will be described through reference to FIGS. 7A and 7B.

An oblique endoscope 32 (see FIG. 7A) inserted into the retractor 31 is usually fixed to an attachment (not shown) that is integrated with the retractor 31, and this restricts movement in the peripheral direction within the retractor 31.

As shown in FIG. 7A, if we assume that the oblique endoscope 32 has been rotated along with the attachment, and if we let dr be the length of the retractor 31 and de the insertion depth of the oblique endoscope 32 in the retractor 31 as shown in FIG. 7B, then the rotation matrix RΘ after a rotation of an angle Θ is calculated with respect to the axis Rz in the depth direction of the distance Ro from the center of the retractor 31 to the center of the oblique endoscope 32.

Next, since vector RoEo′=RΘ×RoEo, the endoscope distal end position can be calculated from the equation endoscope distal end position Ec=Eo′+Rz*de, where de is the insertion depth of the endoscope.

Consequently, the three-dimensional endoscope distal end position can be calculated by two-dimensional mouse operation.

The insertion depth de of the oblique endoscope 32 can be modified by mouse operation (such as with a mouse wheel).

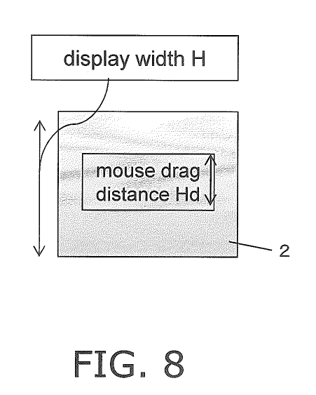

Next, another example related to mapping from two-dimensional input with the mouse 4 to three-dimensional input with an endoscope will be described through reference to FIG. 8.

Usually, an endoscope is connected on the rear end side to a camera head that houses a CCD camera (not shown). The rotation of the display when this camera head is rotated will now be described.

In actual endoscopic surgery, when an image displayed on the display screen of the display 2 ends up being displayed in portrait orientation, just the image is rotated, without changing the field of view, by rotating the camera head so that the orientation of the actual patient will coincide with the orientation of the display on the display 2.

As shown in FIG. 8, to accomplish this by two-dimensional input using the mouse 4, first Θ=360*Hd/H is calculated from the mouse drag distance and the display height.

Then, the rotation matrix R2Θ after a rotation of an angle Θ is calculated with respect to the axis Ry in the depth direction of the center coordinates of the image on the display 2.

If we let U′=R2Θ*U be the new upward vector with respect to the upward vector U of the field of view, the image displayed on the display 2 can be rotated by 90 degrees, for example, without changing the field of view.

This allows the image displayed on the display 2 to be easily adjusted to the same orientation (angle) as the monitor screen in actual endoscopic surgery, by two-dimensional input using the mouse 4.

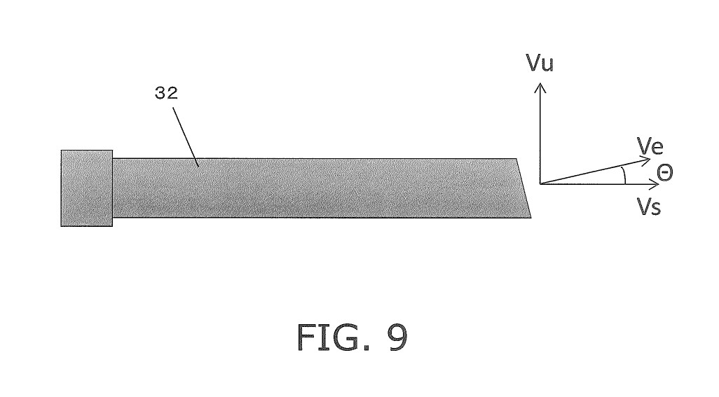

Next, the method for producing a volume rendering image that shows the desired oblique angle of the oblique endoscope 32 will be described through reference to FIG. 9.

In this embodiment, a rotation matrix is applied to the field of view vector according to the oblique angle set for each oblique endoscope 32.

More specifically, first the cross product Vc of the vertical vector corresponding to the oblique direction of the oblique endoscope 32 and the endoscope axis vector Vs corresponding to the axial direction of the retractor 31.

Next, the rotation matrix Rs that undergoes Θ rotation around the Vc is calculated.

Then, the field vector Ve that indicates the oblique angle, can be found as Ve=Rs*Vs.

Consequently, even if the oblique angle varies from one oblique endoscope 32 to the next, the field of view range for each oblique endoscope 32 used in surgery can be set by calculating the field vector Ve on the basis of the information stored in the endoscope parameter storage section 22, etc.

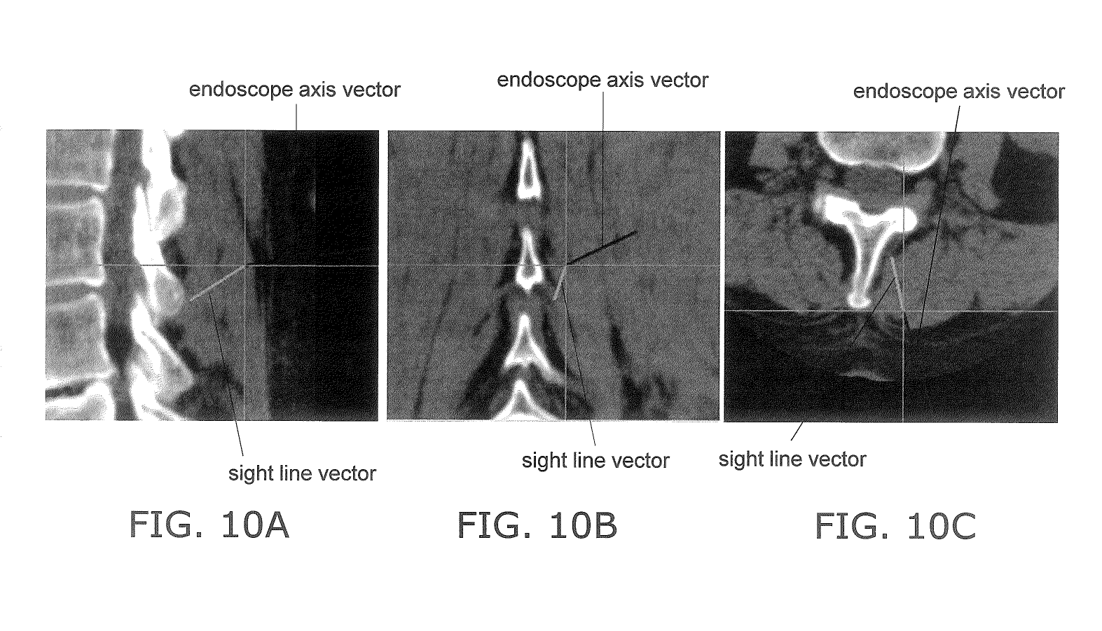

The states when the sight line vector and the distal end position of the oblique endoscope 32 are shown in three-panel view, using the endoscope axis vector Vs and the field vector Ve, are shown in FIGS. 10A to 10C.

As shown in FIGS. 10A to 10C, this allows the insertion direction of the oblique endoscope 32 to be easily ascertained by using a front view (as seen from the side of the patient), a plan view (as seen from the back of the patient), and a side view (as seen from the spine direction of the patient) in a simulation of surgery for lumbar spinal stenosis using the oblique endoscope 32.

With the personal computer 1 in this embodiment, because of the above configuration, an endoscopic image (the endoscopic image display area A1) that shows the restricted display area A2 that is blocked by the retractor 31 is displayed as shown in FIG. 11 in an endoscopic surgery simulation, on the basis of the shape of the retractor 31, the oblique angle and view angle of the oblique endoscope 32, and so forth.

Consequently, a display that approximates the image displayed on the display screen in an actual endoscopic surgery can be displayed by creating a display state that shows the restricted display area A2, which cannot be seen because it is behind the inner wall of the retractor 31 in an actual endoscopic surgery. Therefore, surgery can be assisted more effectively.

Also, in this embodiment, the contact portion between a bone or the like and the retractor 31 is displayed in red, for example, so that the user can tell that the retractor 31 has come into contact with the bone and has reached the insertion limit position in a state in which the retractor 31 has been inserted.

This allows the user to recognize that that the retractor 31 cannot move any deeper. Also, if resection needs to be done at a deeper position, it will be understood that the place where the bone and the retractor are touching will need to be resected. Therefore, it is possible to prevent the endoscopic image from being displayed in simulation at a depth that cannot actually be displayed, so only an image that can be displayed in actual endoscopic surgery can be displayed as the simulation image.

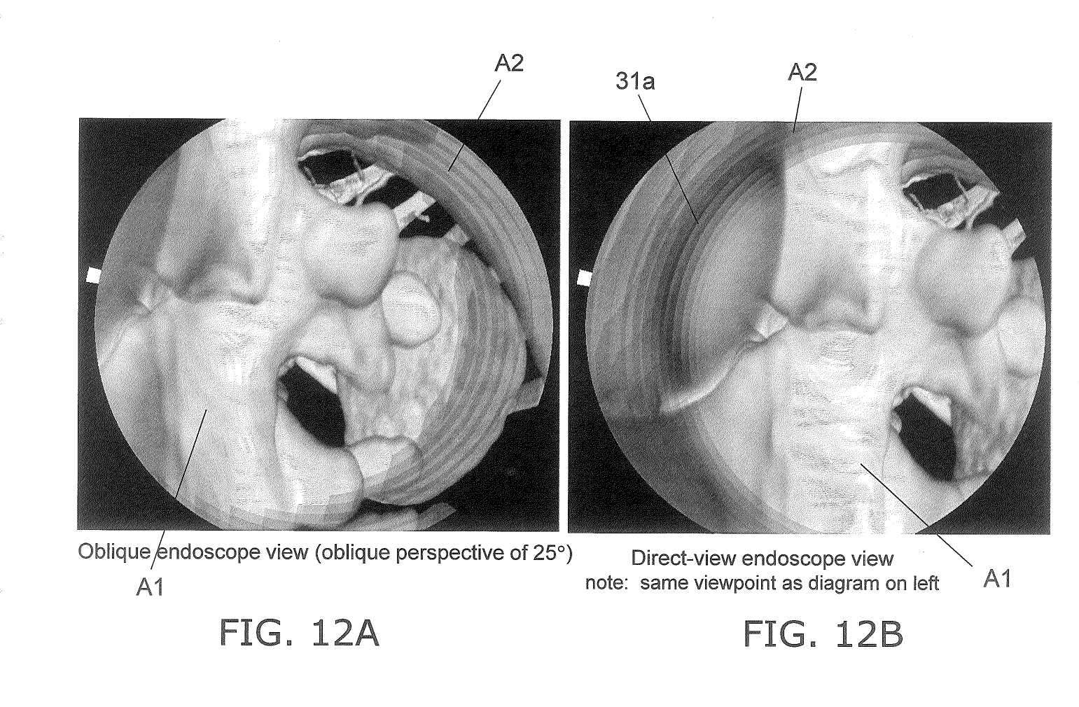

As shown in FIG. 12A, if the oblique angle of the oblique endoscope 32 is 25 degrees, the surgical site will be displayed within the endoscope display area A1 by showing the restricted display area A2 produced by the retractor 31.

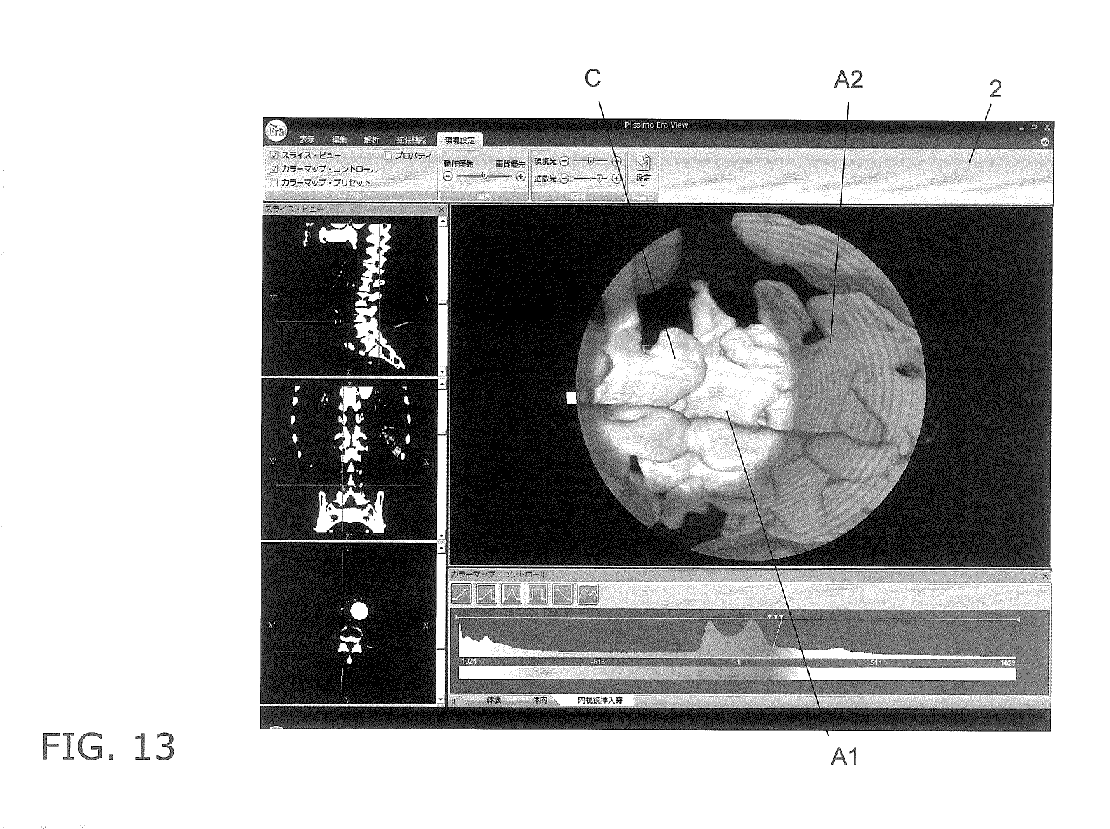

Furthermore, as shown in FIG. 13, the image that is actually displayed on the display 2 of the personal computer 1 in this embodiment can also be combined with the display of a resection target site C or the like, allowing the restricted display area A2 to be shown while displaying the resection target site C within the endoscope display area A1.

### Other Embodiments

An embodiment of the present invention was described above, but the present invention is not limited to or by the above embodiment, and various modifications are possible without departing from the gist of the invention.

(A)

In the above embodiment, an example was described in which the present invention was in the form of a surgery assistance device, but the present invention is not limited to this.

For example, the present invention can be in the form of a surgery assistance program that allows a computer to execute the control method shown in FIGS. 5A and 5B.

(B)

In the above embodiment, an example was described in which the present invention was applied to endoscopic surgery using an oblique endoscope, but the present invention is not limited to this.

As shown in FIG. 12B, for example, the present invention can be applied to the simulation of endoscopic surgery using a direct-view endoscope instead of an oblique endoscope.

FIG. 12B shows the endoscopic image display area A1 and the restricted display area A2 produced with a direct-view endoscope from the same view point as with the oblique endoscope in FIG. 12A.

(C)

In the above embodiment, an example was described in which an image that approximated the endoscopic image displayed during actual surgery was displayed, but the present invention is not limited to this.

For example, a resection simulation device may be combined so that a resection simulation may be carried out while viewing an endoscopic image.

This allows the state during surgery to be reproduced in more detail, allowing the surgery to be assisted more effectively.

(D)

In the above embodiment, an example was described in which surgery for lumbar spinal stenosis was performed, as an example of the surgical simulation using an endoscope pertaining to the present invention, but the present invention is not limited to this.

For example, the present invention may be applied to some other kind of surgery in which an endoscope is used.

(E)

In the above embodiment, an example was described in which surgery for lumbar spinal stenosis was performed using an oblique endoscope, but the present invention is not limited to this.

For example, the present invention may also be applied to surgery in which a direct-view endoscope is used.

(F)

In the above embodiment, an example was described in which an X-ray CT image was used as the tomographic image information for forming a three-dimensional image, but the present invention is not limited to this.

For example, a three-dimensional image may be formed using tomographic image information acquired from a magnetic resonance image (MRI) in which no radiation was used.

### INDUSTRIAL APPLICABILITY

The surgery assistance device of the present invention allows a display to be given that approximates the endoscopic image displayed during actual surgery using an endoscope, so the effect thereof is that surgery can be effectively assisted, and therefore the present invention can be widely applied to various kinds of surgery in which an endoscope is used.

### REFERENCE SIGNS LIST

1 personal computer (surgery assistance device)
2 display (display component)
3 keyboard (input component)
4 mouse (input component)
5 tablet (input component)
6 tomographic image information acquisition section
7 voxel information extractor
8 tomographic image information section
9 memory
10 voxel information storage section
11 voxel label storage section
12 color information storage section
13 volume rendering computer (display controller)
14 voxel label
15 depth sensor
16 bus
17 depth controller
18 voxel label setting section
19 resected voxel label calculation display section
20 window coordinate acquisition section
21 color information setting section
22 endoscope parameter storage section
22a first endoscope parameter storage section
22b second endoscope parameter storage section
22c third endoscope parameter storage section
23 endoscope parameter setting section
24 surgical instrument parameter storage section
24a first surgical instrument parameter storage section
24b second surgical instrument parameter storage section
24c third surgical instrument parameter storage section
25 surgical instrument parameter setting section
26 surgical instrument insertion depth computer
31 retractor (surgical instrument)
32 oblique endoscope (endoscope)
A1 endoscopic image display area (first display area)
A2 restricted display area (second display area)

1. A surgery assistance device configured to display a simulation image during surgery performed by inserting an endoscope into the interior of a surgical instrument, the surgery assistance device comprising:
a tomographic image information acquisition section configured to acquire tomographic image information;
a memory that is connected to the tomographic image information acquisition section and configured to store voxel information related to the tomographic image information;
a volume rendering computer that is connected to the memory and configured to sample voxel information in a direction perpendicular to the sight line on the basis of the voxel information; and
a display controller configured to set a first display area acquired by the endoscope and produced by the volume rendering computer, and a second display area in which display is restricted by the surgical instrument during actual surgery, and display the first and the second display areas on a display section.

2. The surgery assistance device according to claim 1,
further comprising a display section configured to display the first and second display areas.

3. The surgery assistance device according to claim 2,
wherein the display controller detects and displays, as an insertion limit position, the position on the simulation image where the surgical instrument comes into contact with the boundary of the surgical site in a state in which the surgical instrument has been inserted into the body.

4. The surgery assistance device according to claim 1,
wherein the endoscope is an oblique-viewing endoscope.

5. A surgery assistance program configured to display a simulation image during surgery performed by inserting an endoscope into the interior of a surgical instrument, wherein the surgery assistance program is used by a computer to execute a surgery assistance method comprising:
an acquisition step configured to acquire tomographic image information;
a volume rendering step configured to sample voxel information in a direction perpendicular to the sight line on the basis of voxel information related to the tomographic image information; and
a display step configured to set a first display area acquired by the endoscope and produced in the volume rendering step, and a second display area in which display is restricted by the surgical instrument during actual surgery, and display the first and the second display areas on a display section.
